(19) United States
(12) Patent Application Publication
(10) Pub. No.: US 2026/0105983 A1
(43) Pub. Date: Apr. 16, 2026

(54) METHODS FOR COMPUTATIONAL DOCKING OF A SMALL MOLECULE IN A PRION-PROTEIN FILAMENT

(71) Applicant: SB Technology, Inc., Tarrytown, NY (US)

(72) Inventors: Martin Ganahl, Neuberg an der Muerz (AT); Andrea Bortolato, Metuchen, NJ (US); Mary Pitman, Irvine, CA (US); Nicola De Mitri, Cambridge (GB); Maarten Van Damme, Humbeek (BE); Zane Beckwith, Durham, NC (US)

(21) Appl. No.: 19/248,333

(22) Filed: Jun. 24, 2025

Related U.S. Application Data
(60) Provisional application No. 63/667,049, filed on Jul. 2, 2024.

(51) Int. Cl. G16B 15/30; G16B 40/30

(52) U.S. Cl. CPC G16B 15/30; G16B 40/30

(57) ABSTRACT

Methods, systems, and apparatus, including computer programs encoded on a computer storage medium, for computationally docking a small molecule or ligand to a prion-protein In one aspect, a system comprises receiving data comprising a structure of a candidate drug, a structure of a prion-protein filament comprising one or more prions, and a location of a binding site in the prion-protein filament structure, generating a plurality of stacked pose configurations of the candidate drug using at least the candidate drug structure, determining a corresponding docking score for each stacked pose configuration in the plurality of stacked pose configurations in accordance with a measure of a likelihood of binding for the stacked pose configuration in the location of the binding site in the prion-protein filament, and taking an action based on the corresponding docking scores.

### CROSS-REFERENCE TO RELATED APPLICATIONS

This application claims the benefit of priority to U.S. Provisional Application No. 63/667,049, filed on Jul. 2, 2024, the contents of which are herein incorporated by reference.

### BACKGROUND

This specification relates to docking of a molecule, e.g., a small molecule, within a binding site of a protein. In this case, docking refers to computational docking, e.g., algorithms and software that can be used to explore potential binding conformations of a small molecule or ligand within the binding site of the protein. The conformation of a protein-ligand interaction describes the spatial arrangement of the atoms in both the molecule and the protein at the time of binding. In particular, proteins often undergo conformational changes, e.g., induced fit, domain rearrangement, allosteric regulation, etc., when binding to other molecules.

Conventional methods for molecular docking often involve the use of conformational sampling, e.g., by performing an optimized search, e.g., using genetic algorithms or Monte Carlo methods, to sample and evaluate different possible docking conformations for the ligand and protein. For example, a favorable conformation can involve a spatial arrangement of atoms in both the ligand and the protein at the time of binding that lowers the overall energy of the docked protein-ligand system. The choice of molecular docking method often depends on the nature of the protein-ligand interaction.

This specification also relates to prion-proteins (“prions”). Prions are mal-conformed proteins which aggregate into stacks referred to as filaments by transmitting their misfolded conformation to other proteins. Aggregates of such filaments can accumulate over time in human nerve tissue, causing substantial harm to neurons and eventually leading to various fatal clinical endpoints like Multisystematrophie (MSA) or Alzheimer's disease.

### SUMMARY

This specification describes a system implemented as computer programs on one or more computers in one or more locations that can perform docking of a small molecule or ligand to a prion-protein (“prion”). In certain embodiments, the system can perform computational docking of candidate drug compounds with a stacking mode of action to prion filaments. In such embodiments, a stacking mode of action refers to drugs that can bind to themselves and aggregate in stacks. In this specification, a stacked pose configuration of a particular candidate compound refers to a spatial arrangement of one or more of the particular candidate compound stacked together.

Recent research has revealed the potential to inhibit a target prion through modulation with a small-molecule drug compound, e.g., by changing the conformation of the filament through the binding of the drug compound. In particular, a system described in this specification can perform structure-aided drug design to identify candidate drug compounds that strongly bind to the filament with high specificity and one can perform further experimentation to determine whether the drug compound binds in a manner that prevents the filament from aggregating more mal-conformed proteins, e.g., by preventing the filament from transmitting the misfolded conformation of the prions to other proteins or by preventing the filament from binding to the other proteins.

More specifically, certain embodiments of a system described in this specification can leverage the stacking mode of drug compounds with one or more aromatic rings to dock the stacked pose configuration to the binding site of the filament. In this specification, an aromatic ring can refer to a planar cyclic hydrocarbon structure including single and double bonds with delocalized pi electrons, e.g., in a conjugated pi electron system. Generally, prion filaments have tunnel-like binding sites, e.g., the prions aggregate in a manner that forms a hollow area in the filament. In particular, certain embodiments of the system can generate different stacked pose configurations of the candidate compound and determine corresponding docking scores for the stacked pose configurations, e.g., that can be used to evaluate the efficacy of the candidate compound.

According to a first aspect there is provided a method for receiving data comprising a structure of a candidate drug, a structure of a prion-protein filament comprising one or more prions, and a location of a binding site in the prion-protein filament structure, generating a plurality of stacked pose configurations of the candidate drug using at least the candidate drug structure, determining a corresponding docking score for each stacked pose configuration in the plurality of stacked pose configurations in accordance with a measure of a likelihood of binding for the stacked pose configuration in the location of the binding site in the prion-protein filament, and taking an action based on the corresponding docking scores.

In some implementations, receiving data comprising the location of the binding site in the prion-protein filament structure comprises receiving a structure of a reference stack pose indicative of the location of the binding site in the prion-protein filament structure.

In some implementations, receiving data further comprises receiving data comprising a set of configuration parameters.

In some implementations, further comprising preprocessing one or more of the structure of the candidate drug and the structure of the prion-protein filament.

In some implementations, generating the plurality of stacked pose configurations of the candidate drug using at least the candidate drug structure comprises generating a plurality of conformations of the candidate drug structure, wherein each conformation includes at least one aromatic ring structure, combining one or more conformations of the plurality of conformations into a first plurality of stacked pose configurations, and generating the plurality of stacked pose configurations comprising a second plurality of stacked pose configurations using one or more of flipping and rotating the first plurality of stacked pose configurations.

In some implementations, combining the one or more conformations of the plurality of conformations into the first plurality of stacked pose configurations comprises, for each conformation in the one or more conformations, stacking the aromatic ring structure of the first conformation at a first normal distance from a next conformation every second distance along an axis specified using a measure of an opening angle of a cone.

In some implementations, further comprising defining the set of axes using the measure of the opening angle of the cone, wherein defining the set of axes comprises selecting N axes at a specified interval around a unit circle defined by the measure of the opening angle of the cone.

In some implementations, further comprising selecting a subset of the first plurality of stacked pose configurations based at least on a respective measure of energy for each stacked pose configuration.

In some implementations, selecting the subset of the first plurality of stacked pose configurations based at least on the respective measure of energy for each stacked pose configuration comprises determining respective measures of energy for each stacked pose configuration in isolation using a force-field calculation comprising one or more pi-stacking interactions, and selecting a set of M lowest energy stacked pose configurations in isolation based on the respective determined measures of energy.

In some implementations, determining the corresponding docking score for each stacked pose configuration in accordance with the measure of the likelihood of binding for the stacked pose configuration in the location of the binding site in the prion-protein filament comprises, for a number of iterations, optimizing an arrangement of a complex comprising the stacked pose configuration in the location of the binding site in the prion-protein filament in accordance with an energy minimization criteria, determining respective measures of energy for one or more of the arrangement of the stacked pose configuration of the candidate drug with the prion-protein filament, the stacked pose configuration in isolation, and the candidate drug in isolation using one or more force-field functions, wherein each of the force-field functions comprises a corresponding set of force-field parameters, and calculating the docking score for the complex using the determined respective measures of energy.

In some implementations, further comprising providing a final docking score for the complex at a final iteration.

In some implementations, at least one set of the respective sets of force-field parameters have been learned using machine learning.

In some implementations, further comprising using a force-field function comprising one or more pi-stacking interactions for calculating the energy of the stacked pose configuration in isolation, wherein the corresponding set of force-field parameters have been determined using machine learning to account for the one or more pi-stacking interactions.

In some implementations, computing the docking score for the candidate drug using the determined energies comprises calculating a subtraction of one or more of the determined energies as the docking score or calculating a score using Vinardo.

In some implementations, taking the action based on the corresponding docking scores comprises clustering each stacked pose configuration in the plurality of stacked pose configurations using a measure of similarity into a plurality of clusters, for each cluster in the plurality of clusters, identifying a stacked pose configuration as a representative stacked pose configuration of the cluster, and providing the respective docking scores for the representative stacked pose configuration of each cluster.

In some implementations, taking the action based on the corresponding docking scores comprises assessing a measure of efficacy for the candidate drug based on the corresponding docking scores.

In some implementations, the measure of efficacy is determined by aggregating docking scores corresponding with a set of P lowest energy stacked pose configurations in the location of the binding site in the prion-protein filament.

In some implementations, taking the action based on the corresponding docking scores comprises maintaining at least one of the complexes and corresponding docking scores in a database.

In some implementations, taking the action based on the corresponding docking scores comprises using at least one of the complexes and corresponding scores for one or more downstream tasks.

In another aspect, there is provided a system comprising one or more computers and one or more storage devices storing instructions that are operable, when executed by the one or more computers, to cause the one or more computers to perform the method of any one of the example implementation methods described.

In another aspect, there is provided a computer storage medium encoded with a computer program, the program comprising instructions that are operable, when executed by data processing apparatus, to cause the data processing apparatus to perform the method of any one of the example implementation methods described.

Particular embodiments of the subject matter described in this specification can be implemented so as to realize one or more of the following advantages.

Certain embodiments of a system described in this specification can predict a docking score for a candidate compound and prion-protein filament complex. In particular, certain embodiments of a system described in this specification can be configured to explicitly account for the tunnel-like binding site of a prion-protein filament and the stacked conformation of the candidate compound, which is a special docking geometry that is not explicitly accounted for in standard computational docking approaches. By leveraging the stacking mode of candidate drug compounds with an aromatic ring, e.g., pi-stacking, certain embodiments of the system can generate potential stacked pose configurations with high experimental fidelity, i.e., as compared to experimentally verified results, and score each stacked pose configuration in the binding site of the prion-protein filament complex.

In particular, certain embodiments described in this specification can accurately model aromatic interactions between atoms of neighboring candidate compounds required to capture stacked pose configurations and interactions with the prion-protein filament properly, using a specialized force-field calculation. More specifically, such embodiments can leverage an adapted force-field function that incorporates energy adjustments for pi-stacking interactions, e.g., quantum mechanical interactions that are usually roughly approximated by modifying classical forces in standard molecular force-field functions, e.g., the AMBER or SAGE force-field functions.

Additionally, certain embodiments of the system can provide a means of viable computational modeling of stacked pose configurations within the binding site of a prion-protein filament that can be used to further explore how to inhibit the generation or progression of prion filaments. Research targeting prion pathogenesis has been hamstrung by a lack of computational tools specialized for use in prion analysis. Certain embodiments of the system described in this specification can help explore the ability of candidate compounds to inhibit the generation or progression of prion filament accumulation. In other words, such embodiments provide a tool to explore the use of candidate compounds with aromatic rings for the treatment of some of the most fatal neurodegenerative diseases, e.g., Alzheimer's, Parkinson's, Multisystematrophie (MSA), Chronic Traumatic Encephalopathy (CTE), and Creutzfeldt-Jakob disease.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example of a stacked pose configuration of a compound binding to a prion filament.

FIG. 2 is a system diagram of an example prion-protein docking system.

FIG. 3 demonstrates an example method for generating stacked pose configurations of a candidate compound.

FIG. 4 depicts example results of a docking conformation of a stacked pose configuration of a candidate compound predicted using the prion-protein docking system of FIG. 2.

FIG. 5 is an example process for evaluating a docking score for a candidate compound in a prion-protein filament interaction.

FIG. 6 illustrates an example of a computing device and a mobile computing device that can be used to implement the techniques described here.

Like reference numbers and designations in the various drawings indicate like elements.

### DETAILED DESCRIPTION

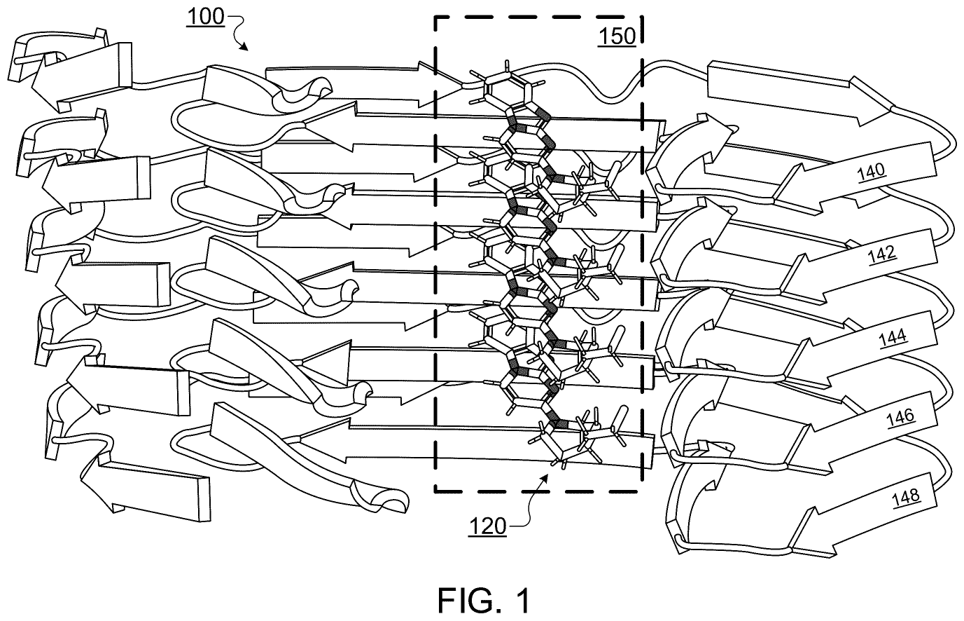

FIG. 1 illustrates an example of a prion-protein (“prion”) filament with a bound drug compound. In particular, FIG. 1 illustrates the stacking of prions to form an aggregated filament and how the stacking mode of the compound allows for a stacked pose configuration to interact and bind with the filament.

Prions are mal-conformed proteins that act as infectious agents by inducing conformational changes in normal proteins, which lead to aggregated filaments, e.g., the filament 100. Prion pathogenesis can lead to many fatal neurogenerative diseases, including rare diseases, e.g., transmissible spongiform encephalopathies, Multisystematrophie (MSA), Chronic Traumatic Encephalopathy (CTE), Creutzfeldt-Jakob disease, and two of the most prevalent age-related diseases: Alzheimer's and Parkinson's disease.

In the particular example depicted, the prion filament 100 is a tau prion-protein filament. Tau proteins and beta amyloid accumulate abnormally in Alzheimer's forming neurofibrillary tangles and amyloid plaque, respectively, inside neurons, and are believed to lead to dementia. It is posited that the abnormal accumulation of both tau proteins and beta amyloid is a prion-led symptom. In this case, the tau prions 140, 142, 144, 146, and 148 have aggregated to form the prion-protein filament 100.

Multiple research efforts are underway to develop treatments for such diseases. A promising approach is the inhibition of a target prion through modulation with a small-molecule drug compound. Certain types of small molecules binding to prions can inhibit the generation or the progression of prion filament accumulation in various cell assays.

The binding modes of some of these drug compounds have been experimentally determined using cryogenic electron microscopy (cryo-EM) imaging, and have revealed a peculiar, stacked binding motif of the drug compound as well, e.g., within a tunnel-like binding site 150 in the protein filament. The stacked pose configuration 120 shows an experimentally determined binding motif of such a small molecule. It is believed that this stacked binding mode is a crucial feature of candidate compounds for treating prion-diseases, and therefore there is a need to develop modern drug docking technology tailored for docking small molecules to prion filaments.

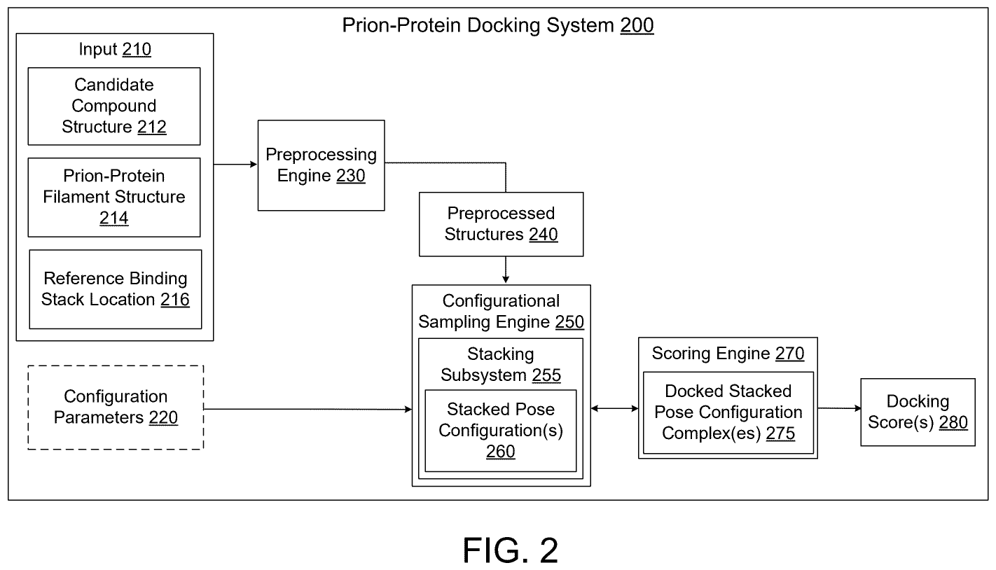

FIG. 2 shows an example embodiment of a prion-protein docking system 200. The prion-protein docking system 200 is an example of a system implemented as computer programs on one or more computers in one or more locations in which the systems, components, and techniques described below are implemented.

In particular, the system 200 can receive one or more inputs 210. In the particular example depicted, the inputs 210 include a candidate compound structure 212, e.g., the small molecule that a user intends to dock in a prion-protein filament, the prion-protein filament structure 214, and the location of a reference binding stack 216, e.g., to specify the location of the tunnel-like binding site of the prion-protein filament. The system 200 can use the inputs to generate a variety of stacked pose configurations and corresponding docking scores, as will be described in more detail below.

As an example, the candidate compound structure 212 and the prion-protein filament structure 214 can include structural data files, e.g., protein databank files, structure data files, or data that represents chemical structures, e.g., Simplified Molecular Input Line Entry System (SMILES) strings. In some cases, the candidate compound structure 212 can be processed as part of a library of a set of candidate compounds.

For example, the location of the reference binding stack 216 can be the location, e.g., the location of the center of mass, of a molecular structure that was previously found to bind with the prion-protein filament, a coordinate location in a grid system, or a relative location based on the amino acids of the prion-protein filament 214. In particular, the system 200 can determine the center of mass of the reference binding stack 216 to provide a reference point as to the active binding site of the prion-protein filament 214, e.g., a reference point for docking. In certain embodiments, a system described in this specification can receive a binding location from a user or from some other source instead of receiving a reference binding stack location 216.

In some cases, the system 200 can receive a set of one or more configuration parameters 220, e.g., parameters directed to controlling performance and runtime aspects of the configurational sampling engine 240, which will be described in more detail below. In particular, the system 200 can receive a set of configuration parameters 220 specified by a user. For example, the parameters 220 can include the number of stacked pose configurations to generate, a time out condition for stacked pose configuration generation, whether the docking should be performed in vacuum or implicit water, and/or a clashing atom distance threshold. As another example, the parameters 220 can include a harmonic restraining force constant, e.g., to ensure atoms or groups of atoms within the stacked pose configuration remain in a desired configuration, number of rotations, or a specified length of the distance between molecules in the stacked pose configuration.

The system 200 can preprocess one or more of the input structures 210, e.g., using a preprocessing engine 230, to generate preprocessed structures 240. In particular the preprocessing engine 230 can prepare the candidate compound structure 212 and the prion-protein filament structure 214 for docking scoring. For example, the system 200 can standardize residue names and compositions, e.g., to replace residues that are broken or incorrectly specified in the corresponding molecular structure data file. As another example, the system 200 can add hydrogen atom(s) where appropriate or otherwise fix the protonation state of molecules. As yet another example, the system can compute partial charges for the molecule.

In some cases, the same preprocessing operations are performed on both the candidate compound structure 212 and the prion-protein filament structure 214. In other cases, different preprocessing operations are performed on each, e.g., the preprocessing engine 230 can protonate the candidate compound structure 212 and add partial charges to the prion-proton filament 214.

The system 200 can then process the preprocessed structures 240 using a configurational sampling engine 240 to generate a variety of stacked pose configurations 260 of the candidate compound 212. The configurational sampling engine 240 can be configured based at least in part on configuration parameters 220. As an example, the configurational sampling engine 240 can generate different conformations by, for example, rotating portions of the candidate drug structure around single bonds, and/or stretching or bending bond angles. As another example, the configurational sampling engine 240 can use data, e.g., from a database, including previously observed conformations of the candidate compound to inform the different conformations.

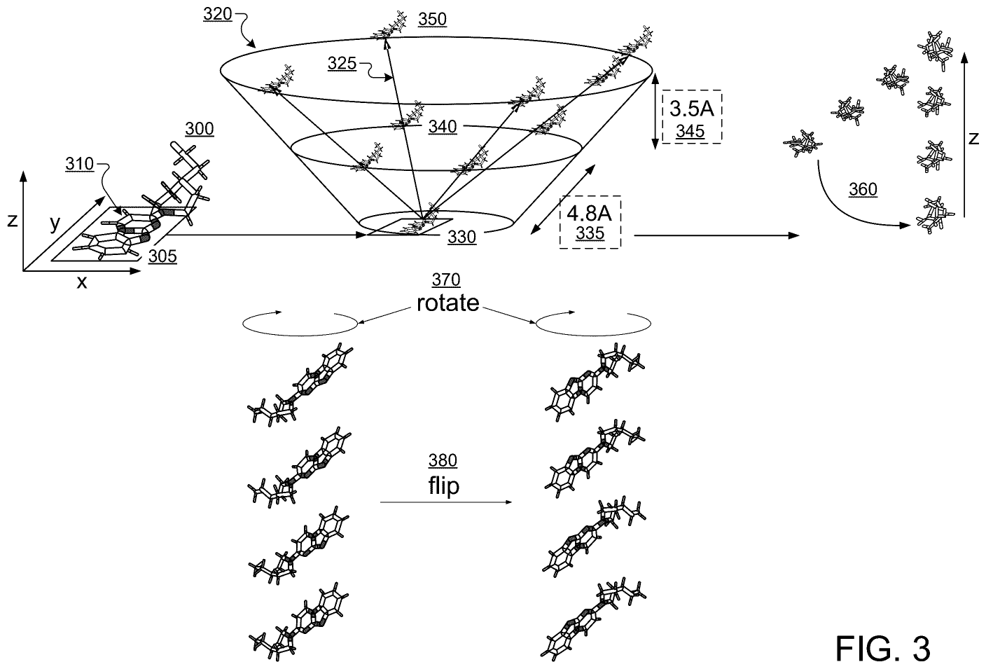

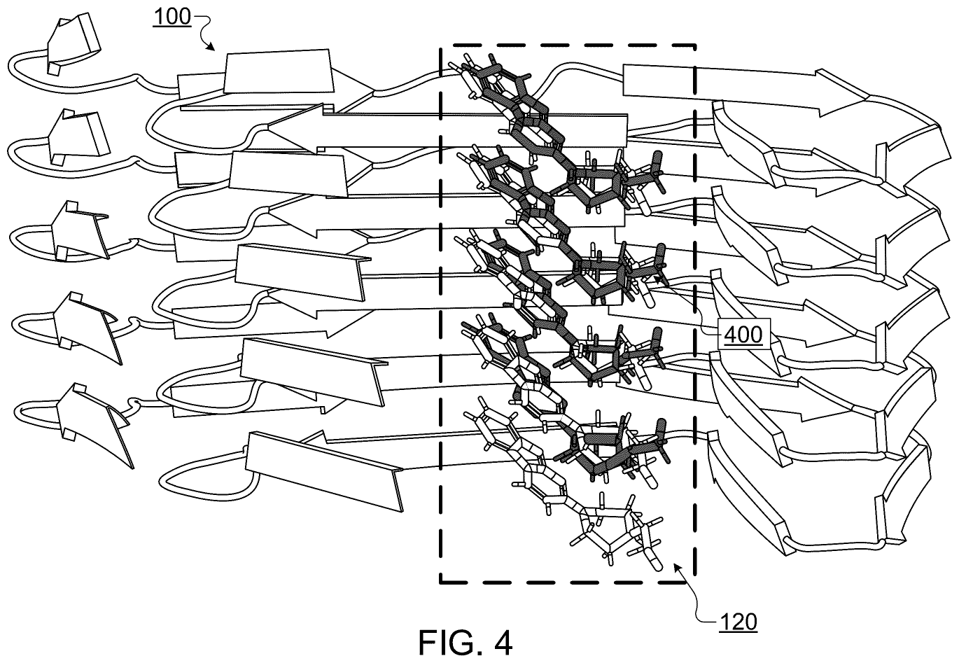

In particular, the configurational sampling engine 240 can generate different conformations of the candidate drug structure and use the different conformations to generate one or more stacked pose configurations 260 of the candidate compound 212, e.g., using a stacking subsystem 255 to stack the different generated conformations of the candidate compound 212. An example for generating the one or more stacked pose configuration(s) 260 by identifying and using the pi-stacking interaction of aromatic rings will be further described with respect to FIGS. 3 and 4. In this case, each candidate compound 212 has at least one aromatic ring, e.g., the system 200 can identify a set of candidate compounds for configurational sampling based on the presence of at least one aromatic ring.

The system 200 can then score the stacked pose configurations 260, e.g., using a scoring engine 270 to determine a measure of docking energy for one or more of the docked stacked pose configurations 260 docked in the prion-protein filament 214, e.g., the docking score(s) 280. More specifically, the system 200 can computationally dock each stacked pose configuration 260 in the one or more stacked pose configurations 260 in the binding site of the protein-prion filament 214, e.g., as determined from the reference binding stack location 216, and determine a docking score 280 for the stacked pose configuration 260 using an energy calculation.

In some cases, the system 200 can determine docking score(s) 280 for each of the generated stacked pose configurations 260. In other cases, the system 200 can determine corresponding docking score(s) 280 for a subset of the generated stacked pose configurations 260. In this case, a user can specify one or more filtering criteria for selecting a subset of the generated stacked pose configurations 260, e.g., energy-based criteria, molecular weight-based criteria, and/or atomic clashing criteria. In particular, the system 200 can receive filtering criteria in the configuration parameters 220 and the system can use the criteria to select a subset of the generated stacked pose configurations 260.

For example, the stacking subsystem 255 can be configured to reject stacked pose configurations 260 that exceed a certain energy threshold in isolation. For example, the system can compute an energy of each of the stacked pose configurations 260 using a force-field computation, e.g., by computing one or more of the van der Waals and electrostatic forces between the candidate compound in the stacked pose configuration either in vacuum or in implicit water. In the case in which the candidate compound 212 has at least one aromatic ring, the force-field calculation can be adapted to account for the pi-stacking repulsion between aromatic rings in each stacked pose configuration 260.

In particular, for each stacked pose configuration in the one or more stacked pose configurations 260, the system 200 can move the stacked pose configuration 260 to the binding site by moving the mean position of the stacked pose configuration to the mean position of the reference binding stack location 216 and can optimize the arrangement of the stacked pose configuration 260 in the location of the binding site. More specifically, the system 200 can use any appropriate optimization algorithm, e.g., gradient descent, genetic algorithms, simulated annealing, etc., to successively update the arrangement of the stacked pose configuration 260 in the location of the binding site in order to minimize the energy of the docked stacked pose configuration complex 275 for a predefined number of iterations. In some cases, the predefined number of iterations can be included in the set of configuration parameters 220. In other cases, the predefined number of iterations can be a system variable of the system 200.

The system 200 can calculate the energy of the one or more docked stacked pose configuration complex(es) 275, e.g., each stacked pose configuration and prion-protein complex, using one or more calculations at each iteration. In this context, the energy of the stacked pose configuration complex(es) 275 is an intermediate docking score for the stacked pose configuration(s) 260 at the iteration. While the examples described below relate to calculating the energy of the docked stacked pose configuration complex(es) 275 using a single example scoring method, the system 200 is not limited to using one scoring method to calculate the intermediate docking scores and can use one or more different methods to calculate the intermediate docking scores across each of the update iterations.

For example, the system 200 can compute a docking energy for the docked stacked pose configuration complex(es) 275 using one or more force-field calculations. In particular, the system 200 can compute one or more of the van der Waals, bond stretching, bond torsion, hydrophobic interactions, and electrostatic forces between the two molecules to determine the energy of the docked stacked pose configuration complex(es) 275.

As an example, the system 200 can calculate a stack-affinity score, e.g., by subtracting the energy of the prion-protein filament 214 in isolation and the stacked pose configuration 260 in isolation from the energy of the docked stacked pose configuration complex 275, e.g., by calculating E_{complex}−E_{filament}−E_{stacked} pose in isolation (where E is the energy). As another example, the system 200 can calculate a ligand-affinity score, e.g., by subtracting energy of the prion-protein filament 214 in isolation and the number of candidate compounds in a stack multiplied by the energy of each individual candidate compound from the energy of the complex, e.g., by calculating E_{complex}−E_{filament}−N_{candidate}*(E_{candidate}).

The system 200 can use one or more force-fields to determine the scores, e.g., one or more force-field calculations to determine the energies of the prion-protein filament 214 in isolation, the stacked pose configuration 260 in isolation, the candidate compound, and the stacked pose configuration 260 and prion-protein complex. As an example, the system 200 can use an open-source force-field calculation, e.g., an AMBER or SAGE force-field calculation, or a user-provided force-field calculation to calculate one or more of the van der Waals, bond stretching, bond torsion, and electrostatic forces between the stacked pose configuration 260 and the prion-protein filament 214 used to calculate the docking score(s) 280. As another example, the system 200 can use a force-field calculation adapted to the pi-stacking interaction of aromatic rings, e.g., in the case that the stacked pose configurations are generated by identifying aromatic rings as will be further described with respect to FIG. 3. In this case, the system 200 can adapt an AMBER, SAGE, or user-provided force-field calculation to account for the pi-stacking interaction of the aromatic rings.

In some cases, the system 200 can use different force-fields, e.g., different force-field types, to calculate the energy of the stacked pose configuration 260 and the energy of the prion-protein filament 214 in isolation. In other cases, the system 200 can use the same force-field type to calculate the energy of the stacked pose configuration 260 and the energy of the prion-protein filament 214 in isolation. As an example, the system 200 can receive the force-field type for the energy of the stacked pose configuration 260, e.g., SAGE, and the energy of the prion-protein filament 214, e.g., AMBER, as part of the set of configuration parameters 220. In this case, SAGE can provide a more accurate energy of the stacked pose configuration 260 relative to AMBER, e.g., since it has received more tuning and parameter improvements than AMBER.

In particular, each force-field calculation can be parameterized by a set of one or more force-field parameters. In some cases, one or more of the parameters of the set of force-field parameters can be learned, e.g., using machine learning. As an example, one or more of the full sets of the force-field parameters used to calculate the energies can be learned to account for classical electromechanical physics interactions expected to arise from the binding interaction, e.g., as opposed to calculating explicitly the energies using a numerical method.

As another example, in the case that the stacked pose configurations 260 are generated using the pi-stacking interaction of aromatic rings, e.g., as will be described with respect to FIG. 3, the system 200 can use an adapted force-field that has been learned, e.g., using machine learning to update the force-field parameters, to account for the quantum mechanical nature of the pi-stacking interaction, e.g., since approximating the quantum mechanical interaction using classical forces is not typically an optimal way to model these interactions.

In this case, the system 200 can use quantum mechanical methods to compute energies for a large number of stacked pose configurations 260 of different stacked pose configuration dimers, e.g., stacked pose configurations including two candidate drug compounds. The system 200 can then configure a correction model to determine one or more values of the force-field parameters, e.g., in order to correct the force-field parameters for the pi-stacking interactions between the two candidate drug compounds in the dimer. As an example, the correction model can be a regression model that can fit the one or more force-field parameters in accordance with minimizing the discrepancy between the predicted energy, e.g., the predicted energy using the force-field without correction, and the quantum mechanically-determined computed energy, for each of the stacked pose configuration dimers.

As yet another example, the system 200 can calculate the energy using an open-source docking program to predict the non-bonded interactions between the prion-protein filament 214 and the stacked pose configuration 260 in the docked stacked pose configuration complex 275. For example, the system 200 can use Vinardo, e.g., as described in Quiroga R, Villarreal M A. “Vinardo: A Scoring Function Based on Autodock Vina Improves Scoring, Docking, and Virtual Screening. ” PLoS One. 2016 May 12; 11(5): e0155183. doi: 10.1371/journal.ponewhich has been optimized using machine learning to predict the interaction energy of a non-bonded interaction, e.g., Gaussian steric attractions, quadratic steric repulsions, Lennard-Jones potentials, etc.

In some cases, the system 200 can analyze the stacked pose configurations 260 to remove any stacked pose configurations that do not satisfy one or more criteria from further optimization. In this case, the system can pare down the number of stacked pose configurations 260 at each iteration or every M iterations. As an example, the system 200 can select the top N configurations based on a measure of likelihood of binding to the prion-protein filament 214, e.g., based on an ordering of the measure of energy of the complex, to advance to the next iteration. As another example, the system 200 can select the configurations with a measure of energy less than a threshold value.

As yet another example, the system 200 can select configurations based on a measure of similarity between each stacked pose configuration 260 and an experimentally observed stacked pose configuration of a candidate drug compound that is known to bind to the prion-protein filament 214. For example, the measure of similarity can be a shape Tanimoto coefficient between the stacked pose configurations 260 and the experimentally observed stacked pose configuration of the compound that is known to bind to the prion-protein filament 214.

After a termination criterion is satisfied, e.g., after the final iteration in the predetermined number of iterations, the system 200 can return a final docking score for each stacked pose configuration complex 275, e.g., the docking score(s) 280. The system 200 can then take an action based on the corresponding docking score(s) 280 for the candidate compound structure 212.

In some cases, the system 200 can use at least one of the stacked pose configurations 260, the docked stacked pose complexes 275, or both and corresponding docking score(s) 280 for one or more downstream tasks. In other cases, the system 200 can maintain at least one of the docked stacked pose complexes 275 and corresponding docking scores in a database, e.g., to store the docked stacked pose complexes 275 and corresponding docking scores 280 for one or more downstream tasks.

As an example, the system 200 can use the corresponding docking score(s) 280 to perform hierarchical clustering as a means of selecting a subset of the stacked pose configurations 260, e.g., for further experimentation, validation, or storage. In particular, the system 200 can cluster generated stacked pose configurations 260 using a measure of similarity, e.g., a measure of similarity between structures, determined features of the stacked pose configuration 260, etc., and can select the stacked pose configuration 260 with the highest score in each respective cluster as the representative stacked pose configuration 260 of the cluster.

As another example, the system 200 can assess a measure of efficacy for the candidate drug based on the corresponding docking scores 280 for a set of conformations for a specific candidate compound. In particular, the system 200 can aggregate, e.g., by computing a mean or median, summing the docking scores, taking the maximum docking score, etc., to generate a global docking score. In some cases, the system 200 can aggregate the docking scores 280 corresponding with the set of M lowest energy stacked pose configuration 260 in the location of the binding site in the prion-protein filament 214.

As yet another example, the docked stacked pose complex 275 and corresponding scores 280 can be provided to one or more users, e.g., a scientist, for target validation, hit generation, and lead optimization as part of a virtual screening campaign. More specifically, the system 200 can be used as a docking tool to down-select, e.g., filter out a subset of candidate drug compounds 212 to treat a certain prion-caused disease from a large library of potential compounds. The down-selected compounds can then be experimentally evaluated, e.g., using binding assays.

As a further example, the stacked pose configurations 260 and corresponding docking scores 280 can be used for a free energy perturbation calculation, e.g., to measure the energy discrepancy between the bound state of the complex 275 and the unbound state of the prion-protein filament 214 structure and the stacked pose configuration 260, or as part of a binding affinity calculation. In particular, the corresponding docking scores 280 can inform the prediction of the strength of the interaction between the stacked pose configuration 260 and the prion-protein filament 214.

FIG. 3 demonstrates an example method for generating stacked pose configurations of a candidate compound using the pi-stacking interaction of aromatic rings.

In this case, certain embodiments can receive or select candidate compounds with at least one aromatic ring for processing, e.g., the candidate compound 300 which has one aromatic ring. An aromatic ring is a cyclic carbon structure with single and double bonds that support a conjugated electron system in which electrons of each atom in the ring are delocalized as a pi cloud above or below the plane of the atoms connected in the aromatic ring. The delocalized pi cloud can lead to pi-pi stacking, e.g., a noncovalent attractive interaction between the pi cloud of two or more aromatic rings. In particular, pi stacking can lead to the parallel alignment of aromatic rings, e.g., forming aromatic ring stacks.

In particular, certain embodiments can identify the aromatic ring structure 310 of the candidate compound 300, e.g., using the stacking subsystem 255 of FIG. 2. As an example, certain embodiments of the system can rotate the aromatic ring to be in a reference plane, e.g., the xy plane 305, and start stacking one or more candidate compounds along one or more stacking axes, e.g., axes defined relative to the location of the reference plane. In the case that no aromatic rings are present, certain embodiments of the system can discard the candidate compound and process a new candidate compound, e.g., from a library of candidate compounds.

In particular, certain embodiments can define a cone with a fixed opening angle intersected by the reference plane, e.g., the cone 320, and sample N vectors, e.g., 4, 10, 20, etc., around the cone to define the axes of stacking. For example, the number N can be a parameter, e.g., a user-defined parameter, included in the configuration parameters, e.g., the configuration parameters 220 of FIG. 2. In the particular example depicted, a certain embodiment of the system has defined four axes of stacking along the cone 320. In some cases, the axes of stacking can be defined at uniform equidistant distances around a unit circle defined by the opening angle of the cone. In other cases, the axes of stacking can be defined by randomly sampling the number N vectors around the opening angle of the cone.

As an example, a certain embodiment of the system can define one or more distances of stacking for stacking the aromatic ring structure of the first candidate compound with the aromatic ring structure of the next candidate compound structure. These distances can be used to generate the stacked pose configurations along each axis of stacking. In the particular example depicted, the system has defined a normal distance 335 between each aromatic ring structure, e.g., 3.5 angstroms, and a rigid translation distance 345 along each axis of stacking, e.g., 4.8 angstroms.

For example, along the stacking axis 325, the system has stacked the candidate compound 340 a normal distance of 3.3 angstroms away from the first candidate compound 330 and a rigid translation distance of 4.8 angstroms along the axis of stacking 325. Likewise, the system has stacked the next candidate compound 350 a normal distance of 3.3 angstroms away from the first candidate compound 340 and a rigid translation distance of 4.8 angstroms along the axis of stacking 325.

As an example, a certain embodiment of the system can receive the one or more distances of stacking from a user as part of the configuration file. In some cases, certain embodiments can receive a value for the stacking distances. In other cases, certain embodiments can receive a range of allowable values for each of the stacking distances. For example, certain embodiments can receive a normal distance range of 3.3 to 3.7 angstroms. As another example, certain embodiments can be configured to operate with predefined system variable(s) defining the distances of stacking.

In some cases, the predefined stacking distances can be informed by known biophysical or biochemical interactions of the candidate compound, e.g., the normal distance and rigid translation distance can be informed by experimentally observed pi-stacking distances, e.g., in a laboratory setting. In the particular example depicted, the normal distance 345, e.g., 3.5 angstroms, and rigid translation distance 335, e.g., 4.8 angstroms, along the stacking axes were verified in a laboratory setting as hallmark distances of the pi-stacking of aromatic ring structures.

Certain embodiments can generate the stacked pose configuration along each of the stacking axes in the cone, e.g., using the one or more stacking distances to determine the placement of the next candidate compound relative to the most recently places candidate compound. In particular, some embodiments can stack the candidate compound a specified number of times, e.g., with a matching criterion based on the number of prions stacked in the filament.

Some embodiments can repeat the generation of stacked pose configurations with a different cone, e.g., such embodiments can repeat the process for stacking with one or more different cones, e.g., cones with different opening angles. As an example, certain embodiments can receive one or more different opening angles from the parameters in the configuration file. As another example, certain embodiments can be configured to operate with a predefined number of opening angles, each parameterizing a different cone.

After generating a number of stacked pose configurations using the stacking axes and one or more distances of stacking, certain embodiments can generate one or more permutations of each stacked pose configuration. In particular, such embodiments can then rotate each stacked pose configuration to align a vertical axis of the stacked pose configuration with a reference axis, e.g., the z-axis as demonstrated by rotation 360. Certain embodiments can then generate one or more permutations of each aligned stacked pose configuration, e.g., by rotating and flipping the stacked pose configurations.

In the particular example depicted, certain embodiments can rotate 370 the stacked pose configuration based on one or more angles around the z-axis and can flip 380 the stacked pose configuration over different axes, e.g., the y-axis. In some cases, the angles of rotation around the z-axis can be predefined. In some cases, the different axes of flipping can be predefined, e.g., in accordance with one or more user-specified parameters defined in the configuration file specifying flipping over the y-axis, the x-axis, or a user-defined axis. As an example, certain embodiments can receive a configuration parameter M defining a set interval of rotation as 2π/M. In other cases, the angles of rotation and different axes of flipping can be randomly sampled from a set of possible angles of rotation and a list of different axes of flipping.

Some embodiments can perform a selection step of the stacked pose configurations, e.g., before generating the permutations of each stacked pose configuration. In particular, such embodiments can select a subset of the generated stacked pose configurations based on a respective measure of energy for each stacked pose. More specifically, such embodiments can select a subset of generated stacked pose configurations with relatively favorable energies such as by selecting the top M most energetically favorable, e.g., lowest energy, stacked pose configurations. As another example, certain embodiments can select the top M%, e.g., top 10%, 15%, 30%, etc., most energetically favorable, e.g., lowest energy, stacked pose configurations. In this case, such embodiments can determine the energy of each stacked pose configuration in isolation, e.g., using a force-field calculation adapted for calculating the energy of one or more pi-stacking interactions.

As yet another example, certain embodiments can perform a selection step by filtering out stacked pose configurations with one or more problematic features. In particular, such embodiments can remove stacked pose configurations with clashing or near clashing atoms, e.g., atoms that are deemed too close based on an identified overlap between their atomic orbitals resulting in a repulsive interaction, e.g., steric hindrance, van der Waals repulsion, or electrons inhabiting the same quantum state. In particular, certain embodiments can detect clashes using a clash detection algorithm, e.g., a geometric overlap detection algorithm, a distance-based detection algorithm, constraint-based solver, etc.

The generated stacked pose configurations can then be scored, e.g., using the scoring engine 270 of FIG. 2. In particular, certain embodiments can compute one or more of the van der Waals, bond stretching, bond torsion, and electrostatic forces between the two molecules to determine the energy of the stacked pose configuration and prion-protein complex to determine a score for the stacked pose configurations, as detailed in FIG. 2.

FIG. 4 depicts example results of a docking conformation of a stacked molecule predicted using the prion-protein docking system 200 of FIG. 2. In particular, FIG. 4 shows the best-scoring generated stacked pose configuration 400 obtained using the prion-protein system 200 of FIG. 2, as depicted in dark gray, compared to an experimentally verified stacked pose configuration for a candidate compound for the tau prion filament, e.g., the prion filament 100 of FIG. 1, as depicted in white.

More specifically, the arrangement of the generated stacked pose configuration was optimized in the binding site of the prion-protein filament over a number of optimization iterations resulting in the best-scoring generated stacked pose configuration 400, which substantially overlaps with the experimentally verified stacked pose 120. In this case, the experimentally verified stack pose 120 was determined using cryo-EM imaging. In particular, the root mean square distance between the two stacked pose configurations 400 and 120 is below 1.5 angstroms, e.g., within a close distance, thereby demonstrating the accuracy of the docked stacked pose configuration complexes generated using the prion-protein docking system 200.

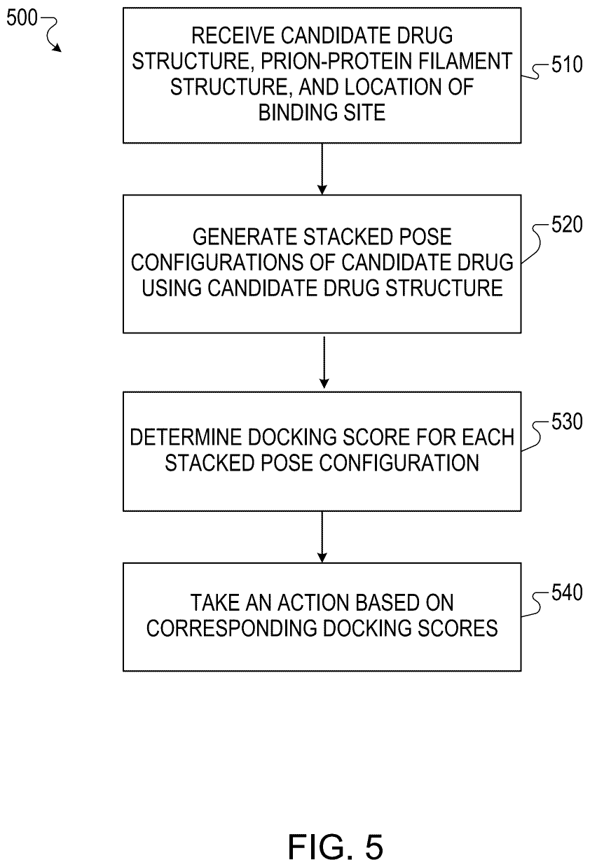

FIG. 5 is a flow diagram of an example process for evaluating a candidate drug in a prion-protein filament interaction. For convenience, the process 500 will be described as being performed by a system of one or more computers located in one or more locations. For example, a prion-protein docking system, e.g., the prion-protein docking system 200 of FIG. 2, appropriately programmed in accordance with this specification, can perform the process 500.

In particular, certain embodiments can receive a candidate drug structure, prion-protein filament structure, and a location of a binding site (step 510). For example, certain embodiments can receive a structural data file that includes the candidate drug structure and the prion-protein filament structure or data that represents chemical structures, e.g., Simplified Molecular Input Line Entry System (SMILES) strings. Certain embodiments can receive the location of a binding site, e.g., a reference binding stack, a coordinate location, etc., and use the location as a reference point for further docking. In some cases, certain embodiments can additionally receive a set of configuration parameters, e.g., parameters directed to adjusting the performance and runtime of the system.

Certain embodiments can generate a number of stacked pose configurations of candidate drug using candidate drug structure (step 520). In particular, certain embodiments can generate a number of conformations of the candidate drug structure, e.g., by using one or more degrees of freedom to vary the arrangement of the atoms in the candidate drug structure. Certain embodiments can then combine one or more of the conformations into a number of stacked pose configurations. In some cases, certain embodiments can generate permutations of the stacked pose configurations, e.g., by flipping and rotating the generated stacked pose configurations.

For example, in the case that the candidate drug structure has at least one aromatic ring structure, certain embodiments can stack the aromatic ring structure at a normal distance from the aromatic ring structure of the next candidate drug along an axis of stacking defined along the opening angle of a cone, e.g., by selecting N axes at a specified interval around a unit circle defined by the measure of the opening angle of the cone. More specifically, certain embodiments can define a normal distance between each aromatic ring structure and a rigid translation distance along each axis of stacking. In some cases, certain embodiments can select a subset of the generated stacked pose configurations based on a respective measure of energy for each stacked pose, e.g., by selecting the stacked pose configurations with the most favorable, e.g., lowest, energies.

Certain embodiments can then determine a docking score for each stacked pose configuration (step 530). In particular, for each stacked pose configuration, certain embodiments can move the stacked pose configuration to the binding site, e.g., by moving the mean position of the reference stack to the mean position of the reference stack, and can optimize the arrangement of the stacked pose configuration in the location of the binding site in the prion-protein filament, e.g., by using gradient descent to minimize the energy of the docked stacked pose configuration over a number of iterations. In some embodiments, the system can remove any stacked pose configurations that do not satisfy one or more criteria from further optimization at a next iteration.

Certain embodiments can determine the docking score by computing the energy of the docked stacked pose configuration complex, e.g., the docked stacked pose configuration in the prion-protein filament binding site. As an example, certain embodiments can compute and subtract one or more of one or more of the energy of the prion-protein filament in isolation, the stacked pose configuration in isolation, the candidate compound in isolation, and the docked stacked pose configuration and prion-protein complex using one or more force-field calculations, e.g., AMBER, SAGE, a user-defined force-field, etc. In the case that the candidate drug structure has at least one aromatic ring structure, the one or more force-field calculations can be adapted to account for the pi-stacking interaction of the aromatic rings. As another example, certain embodiments can calculate the docking score using Vinardo, e.g., as described in “Vinardo: A Scoring Function Based on Autodock Vina Improves Scoring, Docking, and Virtual Screening”, an optimized method for predicting the interaction energy of a non-bonded interaction. In some cases, certain embodiments can calculate a docking score using different methods at different iterations, e.g., the system can calculate a docking score using Vinardo in a first iteration or a first set of iterations, and then calculate a docking score using a subtraction of the energies of the isolated prion-protein filament, the stacked pose configuration in isolation, the candidate compound in isolation, and the docked stacked pose configuration and prion-protein complex in a subsequent iteration or a subsequent set of iterations.

Certain embodiments can then take an action based on the corresponding docking scores (step 540), e.g., by using the stacked pose configurations, docked stacked pose configuration complexes, or both and corresponding docking scores for one or more downstream tasks or by storing the docked stacked pose complexes and corresponding docking scores in a database, e.g., for later use in a downstream task. For example, certain embodiments can assess a measure of efficacy for the candidate drug based on the corresponding docking scores. In particular, certain embodiments can aggregate the corresponding docking scores, e.g., by computing a mean or median, summing the docking scores, taking the maximum docking score, to generate global docking score for the candidate compound.

As another example, the stacked pose configurations and corresponding docking scores can be provided to one or more users, e.g., scientists as part of a virtual screening campaign. As another example, the stacked pose configuration and corresponding scores can be used for a free energy perturbation calculation to determine a measure of binding affinity. As yet another example, certain embodiments can use the corresponding docking scores to perform hierarchical clustering as a means of selecting a subset of the stacked pose configurations, e.g., for further experimentation, validation, or storage.

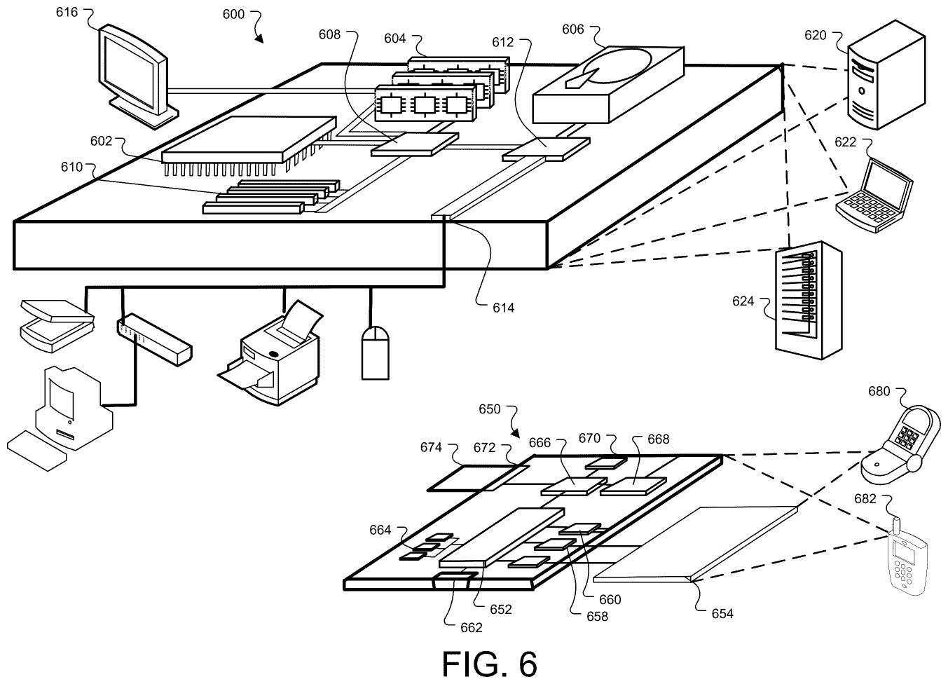

FIG. 6 shows an example of example computer device 600 and example mobile computer device 650, which can be used to implement the techniques described herein. For example, a portion or all of the operations for selecting font variation parameters, generating a custom design variation font file, etc. may be executed by the computer device 600 and/or the mobile computer device 650. Computing device 600 is intended to represent various forms of digital computers, including, e.g., laptops, desktops, workstations, personal digital assistants, servers, blade servers, mainframes, and other appropriate computers. Computing device 650 is intended to represent various forms of mobile devices, including, e.g., personal digital assistants, tablet computing devices, cellular telephones, smartphones, and other similar computing devices. The components shown here, their connections and relationships, and their functions, are meant to be examples only, and are not meant to limit implementations of the techniques described and/or claimed in this document.

Computing device 600 includes processor 602, memory 604, storage device 606, high-speed interface 608 connecting to memory 604 and high-speed expansion ports 610, and low-speed interface 612 connecting to low-speed bus 614 and storage device 606. Each of components 602, 604, 606, 608, 610, and 612, are interconnected using various busses, and can be mounted on a common motherboard or in other manners as appropriate. Processor 602 can process instructions for execution within computing device 600, including instructions stored in memory 604 or on storage device 606 to display graphical data for a GUI on an external input/output device, including, e.g., display 616 coupled to high-speed interface 608. In other implementations, multiple processors and/or multiple busses can be used, as appropriate, along with multiple memories and types of memory. Also, multiple computing devices 600 can be connected, with each device providing portions of the necessary operations (e.g., as a server bank, a group of blade servers, or a multi-processor system).

Memory 604 stores data within computing device 600. In one implementation, memory 604 is a volatile memory unit or units. In another implementation, memory 604 is a non-volatile memory unit or units. Memory 604 also can be another form of computer-readable medium (e.g., a magnetic or optical disk. Memory 604 may be non-transitory.)

Storage device 606 is capable of providing mass storage for computing device 600. In one implementation, storage device 606 can be or contain a computer-readable medium (e.g., a floppy disk device, a hard disk device, an optical disk device, or a tape device, a flash memory or other similar solid state memory device, or an array of devices, such as devices in a storage area network or other configurations.) A computer program product can be tangibly embodied in a data carrier. The computer program product also can contain instructions that, when executed, perform one or more methods (e.g., those described above.) The data carrier is a computer-or machine-readable medium, (e.g., memory 604, storage device 606, memory on processor 602, and the like.)

High-speed controller 608 manages bandwidth-intensive operations for computing device 600, while low-speed controller 612 manages lower bandwidth-intensive operations. Such allocation of functions is an example only. In one implementation, high-speed controller 608 is coupled to memory 604, display 616 (e.g., through a graphics processor or accelerator), and to high-speed expansion ports 610, which can accept various expansion cards (not shown). In the implementation, low-speed controller 612 is coupled to storage device 606 and low-speed expansion port 614. The low-speed expansion port, which can include various communication ports (e.g., USB, Bluetooth®, Ethernet, wireless Ethernet), can be coupled to one or more input/output devices, (e.g., a keyboard, a pointing device, a scanner, or a networking device including a switch or router, e.g., through a network adapter.)

Computing device 600 can be implemented in a number of different forms, as shown in the figure. For example, it can be implemented as standard server 620, or multiple times in a group of such servers. It also can be implemented as part of rack server system 624. In addition or as an alternative, it can be implemented in a personal computer (e.g., laptop computer 622.) In some examples, components from computing device 600 can be combined with other components in a mobile device (not shown), e.g., device 650. Each of such devices can contain one or more of computing device 600, 650, and an entire system can be made up of multiple computing devices 600, 650 communicating with each other.

Computing device 650 includes processor 652, memory 664, an input/output device (e.g., display 654, communication interface 666, and transceiver 668) among other components. Device 650 also can be provided with a storage device, (e.g., a microdrive or other device) to provide additional storage. Each of components 650, 652, 664, 654, 666, and 668, are interconnected using various buses, and several of the components can be mounted on a common motherboard or in other manners as appropriate.

Processor 652 can execute instructions within computing device 650, including instructions stored in memory 664. The processor can be implemented as a chipset of chips that include separate and multiple analog and digital processors. The processor can provide, for example, for coordination of the other components of device 650, e.g., control of user interfaces, applications run by device 650, and wireless communication by device 650.

Processor 652 can communicate with a user through control interface 658 and display interface 656 coupled to display 654. Display 654 can be, for example, a TFT LCD (Thin-Film-Transistor Liquid Crystal Display) or an OLED (Organic Light Emitting Diode) display, or other appropriate display technology. Display interface 656 can comprise appropriate circuitry for driving display 654 to present graphical and other data to a user. Control interface 658 can receive commands from a user and convert them for submission to processor 652. In addition, external interface 662 can communicate with processor 642, so as to enable near area communication of device 650 with other devices. External interface 662 can provide, for example, for wired communication in some implementations, or for wireless communication in other implementations, and multiple interfaces also can be used.

Memory 664 stores data within computing device 650. Memory 664 can be implemented as one or more of a computer-readable medium or media, a volatile memory unit or units, or a non-volatile memory unit or units. Expansion memory 674 also can be provided and connected to device 650 through expansion interface 672, which can include, for example, a SIMM (Single In Line Memory Module) card interface. Such expansion memory 674 can provide extra storage space for device 650, or also can store applications or other data for device 650. Specifically, expansion memory 674 can include instructions to carry out or supplement the processes described above, and can include secure data also. Thus, for example, expansion memory 674 can be provided as a security module for device 650, and can be programmed with instructions that permit secure use of device 650. In addition, secure applications can be provided through the SIMM cards, along with additional data, (e.g., placing identifying data on the SIMM card in a non-hackable manner.)

The memory 664 can include, for example, flash memory and/or NVRAM memory, as discussed below. In one implementation, a computer program product is tangibly embodied in a data carrier. The computer program product contains instructions that, when executed, perform one or more methods, e.g., those described above. The data carrier is a computer-or machine-readable medium (e.g., memory 664, expansion memory 674, and/or memory on processor 652), which can be received, for example, over transceiver 668 or external interface 662.

Device 650 can communicate wirelessly through communication interface 666, which can include digital signal processing circuitry where necessary. Communication interface 666 can provide for communications under various modes or protocols (e.g., GSM voice calls, SMS, EMS, or MMS messaging, CDMA, TDMA, PDC, WCDMA, CDMA2000, or GPRS, among others.) Such communication can occur, for example, through radio-frequency transceiver 668. In addition, short-range communication can occur, e.g., using a Bluetooth®, WiFi, or other such transceiver (not shown). In addition, GPS (Global Positioning System) receiver module 670 can provide additional navigation-and location-related wireless data to device 650, which can be used as appropriate by applications running on device 650. Sensors and modules such as cameras, microphones, compasses, accelerators (for orientation sensing), etc. may be included in the device.

Device 650 also can communicate audibly using audio codec 660, which can receive spoken data from a user and convert it to usable digital data. Audio codec 660 can likewise generate audible sound for a user, (e.g., through a speaker in a handset of device 650.) Such sound can include sound from voice telephone calls, can include recorded sound (e.g., voice messages, music files, and the like) and also can include sound generated by applications operating on device 650.

Computing device 650 can be implemented in a number of different forms, as shown in the figure. For example, it can be implemented as cellular telephone 680. It also can be implemented as part of smartphone 682, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor. The programmable processor can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a device for displaying data to the user (e.g., a CRT (cathode ray tube) or LCD (liquid crystal display) monitor), and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such back end, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the engines described herein can be separated, combined or incorporated into a single or combined engine. The engines depicted in the figures are not intended to limit the systems described here to the software architectures shown in the figures.

A number of embodiments have been described. Nevertheless, it will be understood that various modifications can be made without departing from the spirit and scope of the processes and techniques described herein. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

What is claimed is:

1. A computer-implemented method, the method comprising:
receiving data comprising a structure of a candidate drug, a structure of a prion-protein filament comprising one or more prions, and a location of a binding site in the prion-protein filament structure;
generating a plurality of stacked pose configurations of the candidate drug using at least the candidate drug structure;
determining a corresponding docking score for each stacked pose configuration in the plurality of stacked pose configurations in accordance with a measure of a likelihood of binding for the stacked pose configuration in the location of the binding site in the prion-protein filament; and
taking an action based on the corresponding docking scores.

2. The method of claim 1, wherein receiving data comprising the location of the binding site in the prion-protein filament structure comprises receiving a structure of a reference stack pose indicative of the location of the binding site in the prion-protein filament structure.

3. The method of claim 1, wherein receiving data further comprises receiving data comprising a set of configuration parameters.

4. The method of claim 1, further comprising preprocessing one or more of the structure of the candidate drug and the structure of the prion-protein filament.

5. The method of claim 1, wherein generating the plurality of stacked pose configurations of the candidate drug using at least the candidate drug structure comprises:
generating a plurality of conformations of the candidate drug structure, wherein each conformation includes at least one aromatic ring structure;
combining one or more conformations of the plurality of conformations into a first plurality of stacked pose configurations; and
generating the plurality of stacked pose configurations comprising a second plurality of stacked pose configurations using one or more of flipping and rotating the first plurality of stacked pose configurations.

6. The method of claim 5, wherein combining the one or more conformations of the plurality of conformations into the first plurality of stacked pose configurations comprises, for each conformation in the one or more conformations:
stacking the aromatic ring structure of the first conformation at a first normal distance from a next conformation every second distance along an axis specified using a measure of an opening angle of a cone.

7. The method of claim 6, further comprising defining a set of axes using the measure of the opening angle of the cone, wherein defining the set of axes comprises selecting N axes at a specified interval around a unit circle defined by the measure of the opening angle of the cone.

8. The method of claim 5, further comprising:
selecting a subset of the first plurality of stacked pose configurations based at least on a respective measure of energy for each stacked pose configuration.

9. The method of claim 8, wherein selecting the subset of the first plurality of stacked pose configurations based at least on the respective measure of energy for each stacked pose configuration comprises:
determining respective measures of energy for each stacked pose configuration in isolation using a force-field calculation comprising one or more pi-stacking interactions; and
selecting a set of M lowest energy stacked pose configurations in isolation based on the respective determined measures of energy.

10. The method of claim 1, wherein determining the corresponding docking score for each stacked pose configuration in accordance with the measure of the likelihood of binding for the stacked pose configuration in the location of the binding site in the prion-protein filament comprises, for a number of iterations:
optimizing an arrangement of a complex comprising the stacked pose configuration in the location of the binding site in the prion-protein filament in accordance with an energy minimization criteria;
determining respective measures of energy for one or more of the arrangement of the stacked pose configuration of the candidate drug with the prion-protein filament, the stacked pose configuration in isolation, and the candidate drug in isolation using one or more force-field functions, wherein each of the force-field functions comprises a corresponding set of force-field parameters; and
calculating the docking score for the complex using the determined respective measures of energy.

11. The method of claim 10, further comprising providing a final docking score for the complex at a final iteration.

12. The method of claim 10, wherein at least one set of the respective sets of force-field parameters have been learned using machine learning.

13. The method of claim 12, further comprising using a force-field function comprising one or more pi-stacking interactions for calculating the energy of the stacked pose configuration in isolation, wherein the corresponding set of force-field parameters have been determined using machine learning to account for the one or more pi-stacking interactions.

14. The method of claim 10, wherein computing the docking score for the candidate drug using the determined energies comprises:
calculating a subtraction of one or more of the determined energies as the docking score; or
calculating a score using Vinardo.

15. The method of claim 1, wherein taking the action based on the corresponding docking scores comprises:
clustering each stacked pose configuration in the plurality of stacked pose configurations using a measure of similarity into a plurality of clusters;
for each cluster in the plurality of clusters, identifying a stacked pose configuration as a representative stacked pose configuration of the cluster; and
providing the respective docking scores for the representative stacked pose configuration of each cluster.

16. The method of claim 1, wherein taking the action based on the corresponding docking scores comprises:
assessing a measure of efficacy for the candidate drug based on the corresponding docking scores.

17. The method of claim 16, wherein the measure of efficacy is determined by aggregating docking scores corresponding with a set of P lowest energy stacked pose configurations in the location of the binding site in the prion-protein filament.

18. The method of claim 10, wherein taking the action based on the corresponding docking scores comprises:
maintaining at least one of the complexes and corresponding docking scores in a database.

19. The method of claim 10, wherein taking the action based on the corresponding docking scores comprises:
using at least one of the complexes and corresponding scores for one or more downstream tasks.

20. A system comprising one or more computers and one or more storage devices storing instructions that are operable, when executed by the one or more computers, to cause the one or more computers to perform operations comprising:
receiving data comprising a structure of a candidate drug, a structure of a prion-protein filament comprising one or more prions, and a location of a binding site in the prion-protein filament structure;
generating a plurality of stacked pose configurations of the candidate drug using at least the candidate drug structure;
determining a corresponding docking score for each stacked pose configuration in the plurality of stacked pose configurations in accordance with a measure of a likelihood of binding for the stacked pose configuration in the location of the binding site in the prion-protein filament; and
taking an action based on the corresponding docking scores.

21. One or more computer readable media storing instructions that are executable by a processing device, and upon such execution cause the processing device to perform operations comprising:
receiving data comprising a structure of a candidate drug, a structure of a prion-protein filament comprising one or more prions, and a location of a binding site in the prion-protein filament structure;
generating a plurality of stacked pose configurations of the candidate drug using at least the candidate drug structure;
determining a corresponding docking score for each stacked pose configuration in the plurality of stacked pose configurations in accordance with a measure of a likelihood of binding for the stacked pose configuration in the location of the binding site in the prion-protein filament; and
taking an action based on the corresponding docking scores.
